(19) United States
(12) Patent Application Publication
(10) Pub. No.: US 2025/0295740 A1
(43) Pub. Date: Sep. 25, 2025

(54) Neurotoxin Compositions with Increased Efficacy and Effect Duration

(71) Applicant: Evolus, Inc., Newport Beach, CA (US)

(72) Inventor: Rui Avelar, Newport Beach, CA (US)

(21) Appl. No.: 19/106,794

(22) PCT Filed: Aug. 29, 2023
(86) PCT No.: PCT/US2023/073082

§ 371 Date: Feb. 26, 2025

Related U.S. Application Data
(60) Provisional application No. 63/402,609, filed on Aug. 31, 2022.

(51) Int. Cl. A61K 38/48; A61K 38/02; A61K 38/16; A61P 17/00; A61P 25/00; C07K 2/00; C07K 14/33; C12N 9/52

(52) U.S. Cl. CPC A61K 38/4893; A61K 38/02; A61K 38/164; A61P 17/00; A61P 25/00; C07K 2/00; C07K 14/33; C12N 9/52; C12Y 304/24069

(57) ABSTRACT

Disclosed herein are neurotoxin compositions and methods producing an increase in efficacy as well as an extended effect duration.

### CROSS-REFERENCE TO RELATED APPLICATION

This application claims the benefit of, and priority to, U.S. Provisional Patent Application 63/402,609, filed Aug. 31, 2022, the entire contents of which is incorporated by reference herein.

### FIELD

The present specification relates to the formulation and use of neurotoxin compositions with increased duration of effect as well as efficacy.

### BACKGROUND

Clostridial neurotoxins, a family of closely-related bacterial protein toxins, consist of several antigenically distinguishable botulinum neurotoxins (BoNTs) and tetanus neurotoxins (TeNT). BoNTs are secreted from C. botulinum in the form of multimeric complexes, with a set of nontoxic proteins coded for by genes adjacent to the neurotoxin gene. These protein complexes range in size from 300 kDa to 900 kDa. These large protein complexes consist of the 150 kDa neurotoxin moiety and the set of complexing proteins that are made of a nontoxic non-hemagglutinin protein (or neurotoxin binding protein [NBP]) and several hemagglutinin proteins. These are known as neurotoxin-associated proteins (NAPs) and also as “complexing” or “accessory” proteins. Stabilized through noncovalent interactions, NAPs account for ˜70% of the total mass.

In addition to the type A form of BoNT, there are other serologically distinct forms of BoNT that are also produced by the gram-positive bacteria Clostridium botulinum; serotypes B, C, D, E, F and G. Each of these is distinguished by neutralization with type-specific antibodies.

BoNTs induce a paralysis of the muscle. The 150 kDa component has a light chain and a heavy chain with a disulfide bond between them. The toxin is internalized into the nerve cell at the site of the neuromuscular junction, mediated by the heavy chain. Once inside the cell, there is a reduction step between the heavy chain and light chain, then the light chain portion of the 150 kDa component cleaves an anchoring protein that is attached to the cell membrane, SNAP 25. This cleavage prevents the effective docking of vesicles containing acetylcholine (Ach), a necessary step to release the Ach into neuromuscular junction to initiate muscular contracture. Without this release, the muscle cannot contract and becomes relaxed. Botulinum toxin was initially identified after it was the source of an outbreak of food poisoning.

It is believed that the role of the accessory proteins is to protect the active toxin, (the 150 kDa component), as it moves through the highly acidic environment of the stomach (botulism is typically food-borne). Thus, several theories assert that the accessory proteins are either useless or problematic when administered, e.g., they have the potential of inducing an immunogenic response. It is also believed that once in solution, the accessory proteins rapidly dissociate from the 150 kDa molecule, and have no therapeutic value. Often, neurotoxin providers are asked to state the “specific activity” of their BoNT product, a calculation that assumes the only active ingredient in a BoNT is the 150 kDa component.

BoNTs provide effective means for treating conditions both cosmetic and therapeutic. However, limits in efficacy and duration of effect can necessitate higher dosages and more frequent administrations. Therefore, compositions of increased efficacy and effect duration would be desirable.

### SUMMARY

Disclosed methods and compositions provide increased efficacy and duration of effect as compared to current botulinum neurotoxins, and the use thereof in therapeutic and cosmetic applications.

Disclosed methods comprise the administration of at least one Clostridial neurotoxin, for example a BoNT, augmented with (or in combination with) at least one additional NBP or NAP, for example an accessory protein such as Hn-17, Hn-33, or Hn-70, wherein the resulting amount of the NBP or NAP is greater than the amount typically associated with the Clostridial neurotoxin.

Disclosed methods comprise the administration of at least one Clostridial neurotoxin, for example a BoNT, in combination with at least one vasoconstrictor, for example epinephrine.

Disclosed methods comprise the administration of at least one Clostridial neurotoxin, for example a BoNT, in combination a vasoconstrictor and at least one NBP or NAP, for example an accessory protein such as Hn-17, Hn-33, or Hn-70, wherein the amount of the NBP or NAP is greater than the amount typically associated with the Clostridial neurotoxin.

Disclosed methods comprise combining a neurotoxin formulation with a formulation comprising accessory proteins prior to administration.

Disclosed methods comprise combining a neurotoxin formulation with a formulation comprising a vasoconstrictor prior to administration.

Disclosed methods comprise combining a neurotoxin formulation with a formulation comprising accessory proteins with a formulation comprising a vasoconstrictor prior to administration.

Disclosed compositions comprise at least one Clostridial neurotoxin, for example a BoNT such as BoNT/A, augmented with (or in combination with) at least one additional NBP or NAP, for example an accessory protein such as Hn-17, Hn-33, or Hn-70, wherein the resulting amount of the NBP or NAP is greater than the amount typically associated with the Clostridial neurotoxin.

Disclosed compositions comprise at least one Clostridial neurotoxin, for example a BoNT such as BoNT/A, in combination an additional active agent such as a vasoconstrictor, and at least one NBP or NAP, for example an accessory protein such as Hn-17, Hn-33, or Hn-70, wherein the amount of the NBP or NAP is greater than the amount typically associated with the Clostridial neurotoxin.

### BRIEF DESCRIPTION OF THE FIGURES

FIG. 1 shows exemplary injection sites (100, 120, 140) for glabellar line treatments as described herein.

FIG. 2 shows average DAS score of both legs of all the animals in a control cohort plotted against their respective day. Error bars indicate the standard deviation of all the measurements.

FIG. 3 shows average DAS score of both legs of all the animals in a test cohort plotted against their respective day. Error bars indicate the standard deviation of all the measurements. All the mice died on day 20.

### DETAILED DESCRIPTION

BoNT “efficacy” refers to the amount of activity at a point in time. In clinical studies, the primary measure is usually made 30 days after administration, and is measured by the number of responders at that point. Efficacy can be measured using various assays, for example, the amount of SNAP 25 cleavage at a point in time after exposure to a BoNT. There is a correlation between degree of effect and that amount of BoNT used. The more toxin used, the more effect, until a saturation point is reached.

Duration of effect is a different concept, one that measures a given degree of duration of response or effect. Typically, this is measured from the initiation of the effect to the time it is lost. The duration of effect is believed to be a function of the time required for a new SNAP 25 complex to form in the cell, to facilitate the attachment to the Ach containing vesicles, and release of the Ach into the neuromuscular junction.

Thus, the difference between efficacy and duration can be summarized:
a. Efficacy—how may responders at a time point?
   i. E.g., 1 pt improvement—85% at 1 hour
b. Duration—how long does a response last for?
   i. E.g., 1 pt improvement lasted 2 weeks

Duration of effect for most neurotoxin administrations is typically 3 to 4 months, though it can differ from indication to indication—in the glabellar region, 3 to 4 months is typical, while bladder treatments can result in longer duration of effect.

Extent of effect duration is also a function of how it is measured. Typically a Kaplan Meier analysis is done, and duration is a function of when that response starts, and when it ends. The end can be defined by the median, the point at which less than 50% are responders. Some manufacturers have defined duration as when patients return to baseline, but this is not the convention in pharmaceuticals.

The present disclosure is directed toward methods and compositions for increasing the duration of effect of Clostridial, for example, BoNT treatments, as compared to the duration achieved with current methods. For example, in embodiments, the duration of effect of a Clostridial neurotoxin treatment, for example a BoNT treatment, can be increased by, for example, 10%, 20%, 30%, 40%, 50%, 60%, 70%, 80%, 90%, 100%, or more, as compared to the duration achieved with current methods. Treatments can be applied to achieve, for example, a therapeutic or cosmetic effect.

The present disclosure is further directed toward methods and compositions for increasing the efficacy of Clostridial, for example, BoNT treatments, as compared to the efficacy achieved with current methods. For example, in embodiments, the efficacy of a Clostridial neurotoxin treatment, for example a BoNT treatment, can be increased by, for example, 10%, 20%, 30%, 40%, 50%, 60%, 70%, 80%, 90%, 100%, or more, as compared to the efficacy achieved with current methods. Treatments can be applied to achieve, for example, a therapeutic or cosmetic effect.

In embodiments, disclosed compositions comprise increased amounts of at least one NBP or NAP as compared to the amount normally associated with a neurotoxin, for example a BoNT. For example, each BOTOX® 900 kDa BoNT/A complex contains (or is associated with) 3 Hn-33 molecules. Disclosed embodiments comprise augmenting the composition with additional NBPs or NAPs, for example Hn-33 molecules, thus increasing the ratio of Hn-33 molecules relative to the number of Hn-33 molecules normally associated with the 900 kDa complex. The addition of NBPs or NAPs such as accessory proteins provides a formulation that increases efficacy, effect duration, or both, for example by increasing the uptake of the BoNT into the cell. In embodiments, the amount of accessory protein normally associated with the 900 kDa complex is the amount present in the 900 kDa complex's natural form.

Further embodiments comprise compositions comprising at least one vasoconstrictor and a neurotoxin, for example a BoNT. For example, this addition will aid in the intracellular uptake of both Hn-33 molecules and the 900 kDa complex by decreasing the vascular activity in the region of the neuromuscular junction, allowing for more uptake and increasing the duration of the paralysis period. Disclosed compositions comprising a vasoconstrictor can further comprise an increased amount of NBPs or NAPS as compared to standard or naturally-occurring neurotoxins.

Further embodiments comprise methods of use of disclosed compositions.

Further embodiments comprise methods of manufacture of disclosed compositions.

### Definitions

“Administration,” or “to administer” means the step of giving (i.e. administering) a pharmaceutical composition or active ingredient to a subject. The pharmaceutical compositions disclosed herein can be administered via a number of appropriate routes, including intramuscular or subcutaneous routes of administration, such as by injection (using a needle or a needle-less system), topically, or through use of an implant.

“Botulinum toxin” or “botulinum neurotoxin” or “BoNT” means a neurotoxin derived from Clostridium botulinum, as well as modified, recombinant, hybrid and chimeric BoNTs. A recombinant botulinum toxin can have the light chain and/or the heavy chain thereof made recombinantly by a non-Clostridial species. “Botulinum toxin,” as used herein, encompasses the BoNT serotypes A, B, C, D, E, F, G and H. “Botulinum toxin,” as used herein, also encompasses both a BoNT complex (i.e. the 300, 600 and 900 kDa complexes) as well as pure BoNT (i.e. the about 150 kDa neurotoxic molecule), all of which are useful in the practice of the disclosed embodiments. “Botulinum neurotoxin” also encompasses functional (i.e. biologically active) isoforms, homologs, orthologs, paralogs and fragments of BoNT that show at least 50%, at least 60%, at least 70%, at least 80%, at least 90% and up to 60%, up to 70%, up to 80%, up to 90%, or up to 99% sequence identity to the amino acid sequence of wild-type BoNT, such as wild-type BoNT/A or the neurotoxic component of BoNT serotype A1 deposited with the GenBank database under the accession number AAA23262.

“Clostridial neurotoxin” means a neurotoxin produced from, or native to, a Clostridial bacterium, such as Clostridium botulinum, Clostridium butyricum or Clostridium beratti, as well as a Clostridial neurotoxin made recombinantly by a non-Clostridial species.

“Fast-acting” as used herein refers to a BoNT that produces effects in the patient more rapidly than those produced by, for example, a BoNT/A. For example, the effects of a fast-acting BoNT (such as BoNT/E) can be produced within 36 hours.

“Fast-recovery” as used herein refers to a BoNT that whose effects diminish in the patient more rapidly than those produced by, for example, a BoNT/A. For example, the effects of a fast-recovery BoNT (such as BoNT/E) can diminish within, for example, 120 hours, 150 hours, 300 hours, 350 hours, 400 hours, 500 hours, 600 hours, 700 hours, 800 hours, or the like. However, the usual duration of an intramuscular injection of BoNT/A is typically about 3 to 4 months.

“Neuromodulator” means a biologically active molecule with a specific affinity for a neuronal cell surface receptor. “Neuromodulator” includes Clostridial toxins both as pure toxin and as complexed with one to more non-toxin, toxin-associated proteins.

“Neurotoxin” means a biologically active molecule with a specific affinity for a neuronal cell surface receptor. “Neurotoxin” includes Clostridial toxins both as pure toxin and as complexed with one to more non-toxin, toxin-associated proteins.

“Patient” means a human or non-human subject receiving medical or veterinary care.

“Pharmaceutical composition” means a formulation in which an active ingredient can be a Clostridial toxin. The word “formulation” means that there is at least one additional ingredient (such as, for example and not limited to, an albumin [such as a human serum albumin or a recombinant human albumin] and/or sodium chloride) in the pharmaceutical composition in addition to a BoNT active ingredient. A pharmaceutical composition is therefore a formulation which is suitable for diagnostic, therapeutic or cosmetic administration to a subject, such as a human or animal patient. The pharmaceutical composition can be: in a lyophilized or vacuum dried condition, a solution formed after reconstitution of the lyophilized or vacuum dried pharmaceutical composition with saline or water, for example, or; as a solution that does not require reconstitution. As stated, a pharmaceutical composition can be liquid, semi-solid, or solid. A pharmaceutical composition can be animal-protein free.

“Purified BoNT” means a pure BoNT or a BoNT complex that is isolated, or substantially isolated, from other proteins and impurities which can accompany the BoNT as it is obtained from a culture or fermentation process. Thus, a purified BoNT can have at least 95%, and more preferably at least 99% of the non-BoNT proteins and impurities removed.

“Therapeutic formulation” means a formulation that can be used to treat and thereby alleviate a disorder or a disease and/or symptom associated thereof.

“Therapeutically effective amount” or “cosmetically effective amount” means the level, amount or concentration of an agent (e.g. such as a clostridial toxin or pharmaceutical composition comprising a clostridial toxin) needed to treat a disease, disorder or condition without causing significant negative or adverse side effects.

“Treat,” “treating,” or “treatment” means an alleviation or a reduction (which includes some reduction, a significant reduction a near total reduction, and a total reduction), resolution or prevention (temporarily or permanently) of a symptom, disease, disorder or condition, so as to achieve a desired therapeutic or cosmetic result, such as by healing of injured or damaged tissue, or by altering, changing, enhancing, improving, ameliorating and/or beautifying an existing or perceived symptom, disease, disorder or condition.

“Unit” or “U” means an amount of active BoNT standardized to have equivalent neuromuscular blocking effect as a Unit of commercially available BoNT/A (for example, Onabotulinumtoxin A (BOTOX©)).

### Neurotoxin Compositions

Embodiments disclosed herein comprise neurotoxin compositions. Such neurotoxins can be formulated in any pharmaceutically acceptable formulation in any pharmaceutically acceptable form. The neurotoxin can also be used in any pharmaceutically acceptable form supplied by any manufacturer. Disclosed embodiments comprise use of Clostridial neurotoxins.

The Clostridial neurotoxin can be made by a Clostridial bacterium, such as by a Clostridium botulinum, Clostridium butyricum, or Clostridium beratti bacterium. Additionally, the neurotoxin can be a modified neurotoxin; that is a neurotoxin that has at least one of its amino acids deleted, modified or replaced, as compared to the native or wild type neurotoxin. Furthermore, the neurotoxin can be a recombinantly-produced neurotoxin or a derivative or fragment thereof.

Disclosed compositions comprise at least one Clostridial neurotoxin, for example a BoNT, augmented with at least one additional NBP or NAP, for example an accessory protein, wherein the amount of the NBP or NAP is greater than the amount typically associated with the Clostridial neurotoxin.

In embodiments, the accessory protein comprises Hn-33. In embodiments, the amount of accessory protein as compared to the usual amount naturally associated with the neurotoxin can be increased by, for example, 10%, 20%, 30%, 40%, 50%, 60%, 70%, 80%, 90%, 100%, 120%, 140%, 160%, 180%, 200%, 250%, 300%, or more. In embodiments, the accessory protein can be combined with a neurotoxin other than that which it is usually associated. For example, in embodiments comprising a BoNT/E, a BoNT/A Hn-33 can be added to the formulation.

In embodiments, the accessory protein comprises Hn-33. In embodiments, the amount of accessory protein as compared to the usual amount naturally associated with the neurotoxin can be increased by, for example, a 0.5 molar amount (0.5M), 1M, 1.5M, 2M, 2.5M, 3M, or more. In embodiments, the accessory protein can be combined with a neurotoxin other than that which it is usually associated. For example, in embodiments comprising a BoNT/E, a BoNT/A Hn-33 can be added to the formulation.

Disclosed embodiments comprise formulations comprising a neurotoxin, for example a Clostridial neurotoxin such as a BoNT, augmented with at least one additional accessory protein. For example, each BoNT/A is typically associated with three Hn-33 molecules, therefore disclosed embodiments comprising augmented amounts of an accessory protein can comprise formulations wherein the ratio of Hn-33 molecules to toxin molecules is greater than three. In embodiments, the relative molar mixture of neurotoxin to NAP is, for example, 1:1, 1:3, 1:5, 1:10, or the like.

Disclosed compositions comprise at least one Clostridial neurotoxin, for example a BoNT, in combination with at least one vasoconstrictor, for example epinephrine. In further embodiments, the vasoconstrictor can comprise at least one of alpha-adrenoceptor agonists, vasopressin analogs, norepinephrine, phenylephrine (Sudafed PE), dopamine, dobutamine, migraine and headache medications (serotonin 5-hydroxytryptamine agonists or triptans), or the like.

Disclosed compositions comprise at least one Clostridial neurotoxin, for example a BoNT, in combination a vasoconstrictor and at least one additional NBP or NAP, for example an accessory protein, wherein the amount of the NBP or NAP is greater than the amount typically associated with the Clostridial neurotoxin.

In disclosed embodiments, the neurotoxin is formulated in unit dosage form; for example, it can be provided as a sterile solution in a vial or as a vial or sachet containing a lyophilized powder for reconstituting in a suitable vehicle such as saline for injection. In embodiments comprising a vasoconstrictor, the vasoconstrictor can be added to the BoNT composition prior to use.

In embodiments, the BoNT is formulated in a solution containing saline and pasteurized Human Serum Albumin (HSA), which stabilizes the toxin and minimizes loss through non-specific adsorption. The solution can be sterile filtered (0.2 μm filter), filled into individual vials, and then vacuum-dried to give a sterile lyophilized powder. In use, the powder can be reconstituted by the addition of sterile unpreserved normal saline (sodium chloride 0.9% for injection). In an embodiment, BoNT/A is supplied in a sterile solution for injection with a 5-mL vial nominal concentration of 20 ng/mL in 0.03 M sodium phosphate, 0.12 M sodium chloride, and 1 mg/mL HSA, at pH 6.0.

Although the composition may only contain a single type of neurotoxin, for example BoNT/A, disclosed compositions can include two or more types of neurotoxins, which can provide enhanced therapeutic effects of the disorders. For example, a composition administered to a patient can include BoNT/A and BoNT/E. Administering a single composition containing two different neurotoxins can permit the effective concentration of each of the neurotoxins to be lower than if a single neurotoxin is administered to the patient while still achieving the desired therapeutic effects. This type of “combination” composition can also provide benefits of both neurotoxins, for example, quicker effect combined with longer duration.

The composition administered to the patient can also contain other pharmaceutically active ingredients, such as, protein receptor or ion channel modulators, in combination with the neurotoxin or neurotoxins. These modulators may contribute to the reduction in neurotransmission between the various neurons. For example, a composition may contain gamma aminobutyric acid (GABA) type A receptor modulators that enhance the inhibitory effects mediated by the GABAA receptor. The GABAA receptor inhibits neuronal activity by effectively shunting current flow across the cell membrane. GABAA receptor modulators may enhance the inhibitory effects of the GABAA receptor and reduce electrical or chemical signal transmission from the neurons. Examples of GABAA receptor modulators include benzodiazepines, such as diazepam, oxaxepam, lorazepam, prazepam, alprazolam, halazeapam, chordiazepoxide, and chlorazepate. Compositions may also contain glutamate receptor modulators that decrease the excitatory effects mediated by glutamate receptors. Examples of glutamate receptor modulators include agents that inhibit current flux through AMPA, NMDA, and/or kainate types of glutamate receptors.

### METHODS OF USE

Disclosed neurotoxin compositions can be injected into the patient using a needle or a needleless device. In certain embodiments, the method comprises injecting the composition sub-dermally, subcutaneously, intramuscularly, or through superficial intramuscular injections, into the individual. For example, administering may comprise injecting the composition through a 27 gauge needle, 28 gauge needle, 29 gauge needle, 30 gauge needle, 31 gauge needle, 32 gauge needle, and/or a 33 gauge needle. In certain embodiments, the method comprises administering a composition comprising a botulinum toxin type A.

Administration of the disclosed compositions can be carried out by any suitable means, for example by syringe, catheters, needles and other means for injecting. The injection can be performed on any area of the mammal's body that is in need of treatment. The injection can be into any specific area such as epidermis, dermis, fat, muscle, nerve junction, or subcutaneous layer. Disclosed embodiments can comprise avoiding injecting certain areas, for example avoiding injecting at least one of or both of the frontalis and cervical paraspinal muscle regions.

Disclosed methods comprise the administration of at least one Clostridial neurotoxin, for example a BoNT, augmented with at least one additional NBP or NAP, for example an accessory protein, wherein the amount of the NBP or NAP is greater than the amount typically associated with the Clostridial neurotoxin. For example, each BoNT/A is typically associated with three Hn-33 molecules, therefore disclosed embodiments comprise formulations wherein the ratio of Hn-33 molecules to toxin molecules is greater than three. In embodiments, the relative molar mixture of neurotoxin to NAP is, for example, 1:1, 1:3, 1:5, 1:10, or the like.

Embodiments disclosed herein can reduce local muscular activity and thereby reduce the appearance of cosmetic imperfections or irregularities, for example facial lines. In embodiments the cosmetic irregularities can comprise glabellar lines, forehead lines, “bunny” lines, smile irregularities, chin irregularities, platysmal bands, “marionette” lines, lip lines, crow's feet, eyebrow irregularities, combinations thereof, and the like.

Disclosed methods comprise the administration of at least one Clostridial neurotoxin, for example a BoNT, in combination with at least one vasoconstrictor, for example epinephrine.

Disclosed methods comprise the administration of at least one Clostridial neurotoxin, for example a BoNT, in combination a vasoconstrictor and augmented with at least one NBP or NAP, for example an accessory protein, wherein the amount of the NBP or NAP is greater than the amount typically associated with the Clostridial neurotoxin. For example, each BoNT/A is typically associated with three Hn-33 molecules, therefore disclosed embodiments comprise formulations wherein the ratio of Hn-33 molecules to toxin molecules is greater than three. In embodiments, the relative molar mixture of neurotoxin to NAP is, for example, 1:1, 1:3, 1:5, 1:10, or the like.

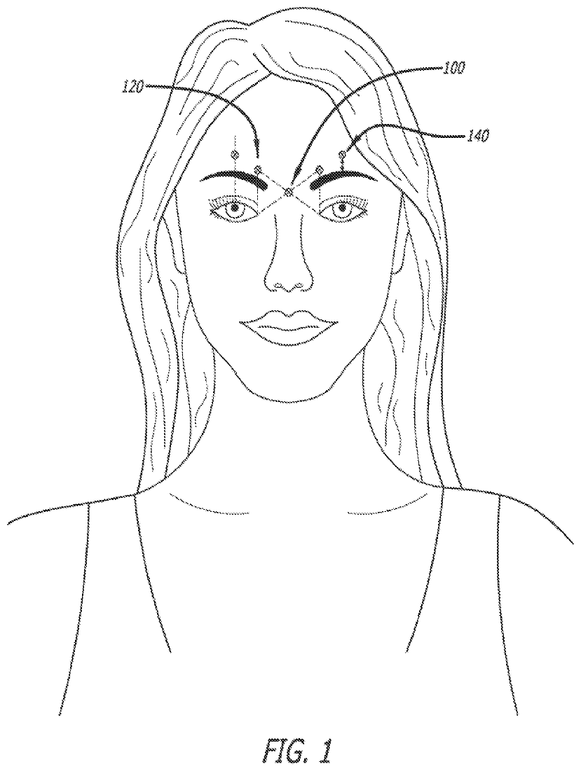

Administration sites useful for practicing the disclosed embodiments can comprise the glabellar complex, including the corrugator supercilli and the procerus; the obicularis oculi; the superolateral fibers of the obicularis oculi; the frontalis; the nasalis; the levator labii superioris aleque nasi; the obicularis oris; the masseter; the depressor anguli oris; and the platysma. Exemplary injection sites useful in glabellar line treatments are shown in FIG. 1. Embodiments comprise treatment of gross wrinkles.

Disclosed embodiments can comprise treatment of, for example, skin disorders, for example, acne, and the like. Disclosed embodiments can comprise treatment of inflammatory skin diseases. For example, disclosed embodiments can comprise treatment of psoriasis, eczema, and the like.

Embodiments comprise methods comprising dermatological surgical procedures, such as treatment for Actinic Keratosis, Seborrheic Keratosis, Basocelular Carcinoma, Squamous Cell Carcinoma, and other lesions or subdermal cysts.

In embodiments, disclosed methods can be used for treating a symptom, disease or condition caused by or associated with hyperactive cholinergic innervation of muscles or exocrine glands in a patient including, but not limited to, dystonia, spasticity, paratonia, diskinesia, focal spasm, strabismus, tremor, tics, migraine, sialorrhea and hyperhidrosis.

Embodiments disclosed herein can reduce local muscular activity and thereby reduce the development of scars, for example scars resulting from surgery. In embodiments the surgery can comprise cosmetic surgery, for example rhinoplasty, an eye lift, a “tummy” tuck, or the like. In embodiments the surgery can comprise other types of medical procedures, for example appendix removal, organ transplant, and the like. In embodiments, methods comprise administering disclosed compositions in proximity to a wound.

Embodiments disclosed herein can reduce local muscular activity and thereby reduce the development of scars, for example scars resulting from trauma. For example, following a traumatic injury, disclosed embodiments can comprise administering disclosed compositions in proximity to trauma, for example a laceration or amputation.

Administration sites useful for practicing disclosed embodiments can comprise any area where muscle activity is to be reduced. For example, disclosed embodiments can include administration to the glabellar complex, including the corrugator supercilli and the procerus; the obicularis oculi; the superolateral fibers of the obicularis oculi; the frontalis; the nasalis; the levator labii superioris aleque nasi; the obicularis oris; the masseter; the depressor anguli oris; and the platysma.

When employed in combination with other surgical procedures, disclosed embodiments can include administration to, for example, muscles of the face, arm, leg, torso, and the like.

Disclosed embodiments can comprise methods for preparing a surgical site prior to the procedure, in order to reduce muscle tension in the proximity of an incision.

Embodiments comprise lower-dose neurotoxin administrations as compared to methods known in the art. For example, the increased efficacy demonstrated by the instant compositions and method can enable lower dosages to achieve the same duration of effect as a higher dosage.

Administration of the disclosed compositions can be carried out by syringe, catheters, needles, needle-less systems, and other means for injecting. The injection can be performed on any area of the mammal's body that is in need of treatment or location that will effect treatment at a desired location, into any specific area such as epidermis, dermis, fat, muscle, nerve junction, or subcutaneous layer.

Disclosed neurotoxin compositions can be injected into the patient using a needle or a needleless device. In certain embodiments, the method comprises sub-dermally injecting the composition in the individual. For example, administering may comprise injecting the composition through a needle of no greater than about 30 gauge. In certain embodiments, the method comprises administering a composition comprising a BoNT, for example a BoNT/A augmented with additional accessory proteins as compared to the amount typically associated with the neurotoxin. The composition can further comprise a vasoconstrictor.

More than one injection and/or sites of injection may be necessary to achieve the desired result. Also, some injections, depending on the location to be injected, may require the use of fine, hollow, Teflon®-coated needles, in certain embodiments guided, for example by electromyography.

The frequency and the amount of injection under the disclosed methods can be determined based on the nature and location of the particular area being treated. In certain cases, however, repeated injection may be desired to achieve optimal results. The frequency and the amount of the injection for each particular case can be determined by the person of ordinary skill in the art.

Although examples of routes of administration and dosages are provided, the appropriate route of administration and dosage are generally determined on a case by case basis by the attending physician. Such determinations are routine to one of ordinary skill in the art (see for example, Harrison's Principles of Internal Medicine (1998), edited by Anthony Fauci et al., 14th edition, published by McGraw Hill). For example, the route and dosage for administration of a Clostridial neurotoxin according to the present disclosed invention can be selected based upon criteria such as the solubility characteristics of the neurotoxin chosen as well as the intensity and scope of the condition being treated.

Disclosed methods can prevent or alleviate the occurrence of pain, nausea, vomiting, light sensitivity, sound sensitivity, and combinations thereof.

In embodiments, methods comprise administering a therapeutically effective amount of at least one neurotoxin to a nerve associated with at least one of the frontalis, corrugator, procerus, masseter, occipitalis, temporalis, trapezius and cervical paraspinal muscle regions.

### Neurotoxin Dosages

The neurotoxin can be administered in an amount of between about 10⁻³ U/kg and about 35 U/kg. In an embodiment, the neurotoxin is administered in an amount of between about 10⁻² U/kg and about 25 U/kg. In another embodiment, the neurotoxin is administered in an amount of between about 10⁻¹ U/kg and about 15 U/kg. In another embodiment, the neurotoxin is administered in an amount of between about 1 U/kg and about 10 U/kg. In many instances, an administration of from about 1 unit to about 300 Units of a neurotoxin, such as a botulinum type A, provides effective therapeutic relief. In an embodiment, from about 5 Units to about 200 Units of a neurotoxin, such as a botulinum type A, can be used and in another embodiment, from about 10 Units to about 100 Units of a disclosed composition can be locally administered into a target tissue.

In embodiments, administration can comprise a total dose per treatment session of about 30 Units of a BoNT, or about 35 Units, or about 40 Units, or about 45 Units, or about 50 Units, or about 55 Units, or about 60 Units, or about 65 Units, or about 70 Units, or about 75 Units, or about 80 Units, or about 85 Units, or about 90 Units, or about 95 Units, or about 100 Units, or about 105 Units, or about 110 Units, or about 115 Units, or about 120 Units, or about 125 Units, or about 130 Units, or about 135 Units, or about 140 Units, or about 145 Units, or about 150 Units, or about 155 Units, or about 160 Units, or about 165 Units, or about 170 Units, or about 175 Units, or about 180 Units, or about 185 Units, or about 190 Units, or about 195 Units, or about 200 Units, or about 205 Units, or about 210 Units, or about 215 Units, or about 220 Units, or about 225 Units, or about 230 Units, or about 235 Units, or about 240 Units, or about 245 Units, or about 250 Units, or about 255 Units, or about 260 Units, or about 265 Units, or about 270 Units, or about 275 Units, or about 280 Units, or about 285 Units, or about 290 Units, or about 295 Units, or about 300 Units, or the like.

In embodiments, administration can comprise a total dose per treatment session of not less than 10 Units of a neurotoxin, for example a BoNT, or not less than 20 Units, or not less than 30 Units, or not less than 40 Units, or not less than 50 Units, or not less than 60 Units, or not less than 70 Units, or not less than 80 Units, or not less than 90 Units, or not less than 100 Units, or not less than 110 Units, or not less than 120 Units, or not less than 130 Units, or not less than 140 Units, or not less than 150 Units, or not less than 160 Units, or not less than 170 Units, or not less than 180 Units, or not less than 190 Units, or not less than 200 Units, or not less than 210 Units, or not less than 220 Units, or not less than 230 Units, or not less than 240 Units, or not less than 250 Units, or not less than 260 Units, or not less than 270 Units, or not less than 280 Units, or not less than 290 Units, or not less than 300 Units, or the like.

In embodiments, administration can comprise a total dose per treatment session of not more than 10 Units of a neurotoxin, for example botulinum type A neurotoxin, or not more than 20 Units, or not more than 30 Units, or not more than 40 Units, or not more than 50 Units, or not more than 60 Units, or not more than 70 Units, or not more than 80 Units, or not more than 90 Units, or not more than 100 Units, or not more than 110 Units, or not more than 120 Units, or not more than 130 Units, or not more than 140 Units, or not more than 150 Units, or not more than 160 Units, or not more than 170 Units, or not more than 180 Units, or not more than 190 Units, or not more than 200 Units, or not more than 210 Units, or not more than 220 Units, or not more than 230 Units, or not more than 240 Units, or not more than 250 Units, or not more than 260 Units, or not more than 270 Units, or not more than 280 Units, or not more than 290 Units, or not more than 300 Units, or the like.

In embodiments, administration can comprise a total dose per year of not more than 400 Units of a neurotoxin, for example botulinum type A neurotoxin, or not more than 500 Units, or not more than 600 Units, or not more than 700 Units, or not more than 800 Units, or not more than 900 Units, or not more than 1000 Units, or the like.

In embodiments, neurotoxin administration, for example botulinum administration, to the nerve associated with at least one of the frontalis, corrugator, procerus, masseter occipitalis, temporalis, trapezius and cervical paraspinal muscle regions can comprise a total dose per treatment session of about 40 Units of a BoNT, or about 50 Units, or about 60 Units, or about 70 Units, or about 80 Units, or about 90 Units, or about 100 Units, or about 110 Units, or about 120 Units, or about 130 Units, or about 140 Units, or about 150 Units, or about 160 Units, or about 170 Units, or about 180 Units, or about 190 Units, or about 200 Units, or about 210 Units, or the like.

In embodiments, the dosage per injection site t can be, for example, 2 units, 3 units, 4 units, 5 units, 10 units, 15 units, 20 units, between 2 and 10 units, between 4 and 8 units, between 4 and 6 units, between 5 and 20 units, between 5 and 15 units, between 5 and 10 units, between 10 and 15 units, or the like.

In embodiments, the dose of the neurotoxin is expressed in protein amount or concentration. For example, in embodiments the neurotoxin can be administered in an amount of between about 0.2 ng and 20 ng. In an embodiment, the neurotoxin is administered in an amount of between about 0.3 ng and 19 ng, about 0.4 ng and 18 ng, about 0.5 ng and 17 ng, about 0.6 ng and 16 ng, about 0.7 ng and 15 ng, about 0.8 ng and 14 ng, about 0.9 ng and 13 ng, about 1.0 ng and 12 ng, about 1.5 ng and 11 ng, about 2 ng and 10 ng, about 5 ng and 7 ng, and the like, into a target tissue such as a muscle.

Ultimately, however, both the quantity of toxin administered and the frequency of its administration will be at the discretion of the physician responsible for the treatment and will be commensurate with questions of safety and the effects produced by the toxin.

Disclosed embodiments comprise treatments that can be repeated. For example, a repeat treatment can be performed when the patient begins to experience symptoms. However, preferred embodiments comprise repeating the treatment prior to the return of symptoms. Therefore, disclosed embodiments comprise repeating the treatment, for example, after 6 weeks, 8 weeks, 10 weeks, 12 weeks, 14 weeks, 16 weeks, 18 weeks, 20 weeks, 22 weeks, 24 weeks, or more. Repeat treatments can comprise administration sites that differ from the administration sites used in a prior treatment. For example, in embodiments, the frontalis can be injected in the initial treatment, then not injected in a following treatment.

A controlled release system can be used in the embodiments described herein to deliver a neurotoxin in vivo at a predetermined rate over a specific time period. A controlled release system can be comprised of a neurotoxin incorporated into a carrier. The carrier can be a polymer or a bio-ceramic material. The controlled release system can be injected, inserted or implanted into a selected location of a patient's body and reside therein for a prolonged period during which the neurotoxin is released by the implant in a manner and at a concentration which provides a desired therapeutic efficacy.

Polymeric materials can release neurotoxins due to diffusion, chemical reaction or solvent activation, as well as upon influence by magnetic, ultrasound or temperature change factors. Diffusion can be from a reservoir or matrix. Chemical control can be due to polymer degradation or cleavage of the drug from the polymer. Solvent activation can involve swelling of the polymer or an osmotic effect.

### Kits

A kit for practicing disclosed embodiments is also encompassed by the present disclosure. The kit can comprise a 30 gauge or smaller needle and a corresponding syringe. The kit can also comprise at least one Clostridial neurotoxin composition, such as a botulinum type A toxin composition. The neurotoxin composition may be provided in the syringe. The composition is injectable through the needle. The kits are designed in various forms based the sizes of the syringe and the needles and the volume of the injectable composition(s) contained therein, which in turn are based on the specific deficiencies the kits are designed to treat.

Disclosed compositions can be provided in pre-loaded syringes.

### Methods of Composition Preparation

Disclosed compositions can be produced by adding at least one NBP or NAP, for example an accessory protein such as Hn-17, Hn-33, or Hn-70, to a botulinum formulation. In embodiments the at least one NBP or NAP is added and then the composition is lyophilized. In embodiments, the at least one NBP or NAP is added just prior to administration.

In embodiments, the at least one NBP or NAP can be naturally-derived or recombinant.

### EXAMPLES

The following non-limiting Examples are provided for illustrative purposes only in order to facilitate a more complete understanding of representative embodiments. This example should not be construed to limit any of the embodiments described in the present specification.

### Example 1

### Treatment of Axillary Hyperhidrosis

A 44-year old male complains of excessive axillary perspiration. His doctor recommends a Minor's starch iodine test. In the Minor's test, the skin is cleaned, shaved, and dried. A 3.5% iodine in alcohol solution is applied to the skin of the affected area, and starch flour is sprinkled on top. The color changes to a dark violet as sweat contacts the iodine-starch mixture. This indicates a positive sweat test and yields a diagram of the distribution of active eccrine glands.

His doctor diagnoses axillary hyperhidrosis, and prescribes injections of BoNT/A in the pattern indicated by the Minor's test to provide relief. The injections are made subcutaneously (s/c) to the underarm in a grid-like pattern approximately every 1-2 cm apart. Each injection contains 10 units of BoNT/A augmented with Hn-33 in an amount 50% greater than typically associated with the neurotoxin.

The patient experiences positive results for six months after the treatment.

### Example 2

### Treatment of Hyperhidrosis in the Feet

A 66-year old male complains of excessive perspiration from the soles of his feet. His doctor diagnoses hyperhidrosis, and prescribes injections of BoNT/E to provide rapid relief. The injections are made subcutaneously to the sole of the foot in a grid-like pattern approximately every 1-2 cm apart. Each injection contains 5 ng of BoNT/E as well as 0.5% epinephrine in lidocaine.

The patient experiences positive results for four months after the treatment.

### Example 3

### Treatment of Hyperhidrosis in the Palms

A 19-year old toxin-naïve male complains of excessive perspiration from his palms. His doctor diagnoses hyperhidrosis, and prescribes injections of BoNT/A to provide rapid relief. The injections are made to the palms in a grid-like pattern approximately every 1-2 cm apart. Each injection contains 10 units of BoNT/A augmented with Hn-33 in an amount 50% greater than typically associated with the neurotoxin. The patient experiences positive results for six months after the treatment.

### Example 4

### Treatment of Crows Feet

A 57 year old man with crow's feet resulting from years of sun exposure seeks treatment from his physician. The physician recommends a composition as disclosed herein, which is injected sub-dermally on either side of the patient's eyes. Each injection contains 5 ng of BoNT/E as well as 1% epinephrine in lidocaine, with several injections made on either side of the eye. The crow's feet disappear within about 2 days after treatment, and remain reduced for five months.

### Example 5

### Treatment for Brow Lift

A 60 year old neurotoxin-naive woman presents with eyebrows extending below her brow bone. The physician recommends a composition as disclosed herein, which is injected subdermally above each eye. Each injection site receives about 10 units of BoNT/A as well as 1% epinephrine in lidocaine, with several injections made on either side of the eye. The drooping of the brow is reduced within about 2 days, and is substantially alleviated for six months after administration.

### Example 6

### Treatment for Breast Augmentation

A 30 year old woman elects breast augmentation surgery. 4 hours prior to the procedure, BoNT/B augmented with Hn-33 in an amount 50% greater than typically associated with the neurotoxin is administered in the proximity of where the surgical incisions will be made. The administration reduces muscle tension in the area of the incision, resulting in minimal scarring.

### Example 7

### Treatment for Breast Reconstruction

A 30 year old neurotoxin-naive woman elects breast reconstruction surgery. 14 hours prior to the procedure, BoNT/F augmented with Hn-33 in an amount 10% greater than is typically associated with the neurotoxin is administered in the proximity of where the surgical incisions will be made. The administration reduces muscle tension in the area of the incision, resulting in minimal scarring. Two weeks after the procedure, a supplemental dose is administered.

### Example 8

### Treatment of Episiotomy

A 44 year old woman undergoes an episiotomy. Immediately after the procedure, BoNT/F augmented with Hn-33 in an amount 10% greater than typically associated with the neurotoxin is administered to the tissue surrounding surgical area. Within 20 hours, muscle and nerve activity surrounding the wound is greatly reduced.

### Example 9

### Treatment of a Sports Hernia

A 28 year old neurotoxin-naive man suffers a sports hernia. His doctor administers 4 ng of BoNT/F augmented with Hn-33 in an amount 100% greater than typically associated with the neurotoxin is administered to the tissue surrounding both the hernia. Within 10 hours, muscle and nerve activity surrounding the injury is greatly reduced.

### Example 10

### Treatment of a Shoulder Separation

A 48 year old man suffers a shoulder separation. His doctor administers BoNT/A augmented with Hn-33 in an amount 20% greater than typically associated with the neurotoxin to the tissue surrounding both the hernia. Muscle and verve activity in the area of the injections is reduced for 6 months.

### Example 11

### Treatment of a Torn ACL

A 23 year old woman suffers a torn ACL. 6 hours after the injury, her doctor administers 10 units of BoNT/A as well as 1% epinephrine in lidocaine to the muscle surrounding the torn ligament. Within 30 hours, muscle and nerve activity surrounding the wound is greatly reduced.

### Example 12

### Treatment of Neuropathic Pain

A 42 year old man experiences neuropathic diabetes pain in his feet. To alleviate the patient's symptoms, about 10 units of BoNT/D as well as 0.7% epinephrine in lidocaine is injected into the vicinity of nerves in the feet. The patient experiences reduced pain for 7 months.

### Example 13

### Treatment of Episiotomy

A 24 year old woman undergoes an episiotomy. Immediately after the procedure, about 20 units of BoNT/A as well as 1% epinephrine in lidocaine to the tissue surrounding surgical area. The BoNT/A is augmented with Hn-33 in an amount 40% greater than typically associated with the neurotoxin.

### Example 14

### Glabellar Line Treatment

A 57 year old man undergoes treatment for glabellar lines. BoNT/A augmented with Hn-33 in an amount 40% greater than typically associated with the neurotoxin as well as 0.7% epinephrine in lidocaine was delivered at 5 injection sites in equal volumes (5 Units, 0.1 mL per site into the procerus, left and right medial corrugators, and left and right lateral corrugators) in a standardized fashion (see FIG. 1). The appearance of the glabellar lines is reduced for 6 months.

### Example 15

### Glabellar Line Treatment

A 27 year old man undergoes treatment for glabellar lines. BoNT/E augmented with Hn-33 in an amount 70% greater than typically associated with the neurotoxin as well as 0.5% epinephrine in lidocaine was delivered at 5 injection sites in equal volumes (5 Units, 0.1 mL per site into the procerus, left and right medial corrugators, and left and right lateral corrugators) in a standardized fashion (see FIG. 1). The appearance of the glabellar lines is reduced for 7 months.

### Example 16

### Glabellar Line Treatment

A 47 year old woman undergoes treatment for glabellar lines. BoNT/B augmented with Hn-33 in an amount 20% greater than typically associated with the neurotoxin was delivered at 5 injection sites in equal volumes (5 Units, 0.1 mL per site into the procerus, left and right medial corrugators, and left and right lateral corrugators) in a standardized fashion (see FIG. 1). The appearance of the glabellar lines is reduced for 5 months.

### Example 17

### Treatment of Chronic Migraine

A chronic migraine patient is treated via injection of 15 U of BoNT/A augmented with Hn-33 in an amount 20% greater than typically associated with the neurotoxin several locations along the trigeminal nerve, including the frontalis, corrugator, procerus, and occipitalis.

The patient reports fewer migraines for 24 weeks following the treatment.

### Example 18

### Treatment of Chronic Migraine

A chronic migraine patient is treated via injection of 20 U of BoNT/E augmented with Hn-33 in an amount 80% greater than typically associated with the neurotoxin at several locations along the trigeminal nerve, including the frontalis, corrugator, procerus, and occipitalis.

The patient reports fewer migraines for 24 weeks following the treatment.

### Example 19

### Treatment of Episodic Migraine

A chronic migraine patient is treated via injection of 15 U of BoNT/A each at several locations along the trigeminal nerve, including the frontalis, corrugator, procerus, masseter, and occipitalis. The cervical paraspinal region is not injected. The patient is also administered 30 U of BoNT/A (15 U bilaterally) intra-orally to the pterygopalatine space.

The patient reports fewer migraines for 16 weeks following the treatment.

### Example 20

### Composition Testing

Recovery experiments in mice after IM administration of different formulations of Evolus sample with Hn-33.

Study Goal—To Evaluate Muscle Weakness by the DAS Scoring and Rota-rod Assay Systems for the Sponsor's Test Articles in Nu/NU mice or Balb/c mice.

| | Test articles will be sent to the animal | |
|---|---|---|
| Dose | facility pre-formulated by the | |
| Formulation | sponsor and ready to be administered. | |
| Animal | | Dose |
| Cohort(n = 3) | Anesthesia | administration |
| 1 | Isoflurane | Negative Control (1X PBS) |
| 2 | | Positive control-I (P1 (in-house |
| | | API): 10 units) |
| 3 | | Positive Control-II (P2 |
| | | (Evolus formulated toxin): 10 |
| | | units) |
| 4 | | Single IM injection of a 1:1 |
| | | molar mixture of two |
| | | proteins in both hind legs |
| | | (Therapeutic Protein P1 + |
| | | additive Native-Hn-33) |
| 5 | | Single IM injection of a |
| | | 1:3 molar mixture of two |
| | | proteins in both hind legs |
| | | (Therapeutic Protein P1 + |
| | | additive Native Hn-33) |
| 6 | | Single IM injection of a |
| | | 1:5 molar mixture of two |
| | | proteins in both hind legs |
| | | (Therapeutic Protein P1 + |
| | | additive Native Hn-33) |
| 7 | | Single IM injection of a |
| | | 1:5 molar mixture of two |
| | | proteins in both hind legs |
| | | (Therapeutic Protein P1 + |
| | | additive Recombinant Hn-33) |
| 8 | | Single IM injection of a |
| | | 1:10 molar mixture of two |
| | | proteins in both hind leg |
| | | (Therapeutic Protein P1 + |
| | | additive Native Hn-33) |
| 9 | | Single IM injection of a |
| | | 1:5 molar mixture of two |
| | | proteins in both hind leg |
| | | (Therapeutic Protein PI (In- |
| | | house API) + additive |
| | | Recombinant Hn-33) |

| | |
|---|---|
| Treatment | Cohort 1-9; Animals will be trained on rotarod. |
| | Cohort 1-9: Animals receive isoflurane on Day 0 |
| | Cohort 1-9: Test articles, positive and negative |
| | control IM injections on gastrocnemius |
| | muscles. |
| | Dose formulations are injected intramuscularly |
| | to both hind limb of each mouse while under |
| | anesthesia. Negative and positive controls are |
| | injected to both hind limb intramuscularly to |
| | the respective cohorts while under anesthesia. |
| | DAS score and rotarod (number of rotations |
| | and seconds they stayed on the rod before |
| | falling down) should recorded for each mouse at |
| | each time points. |
| | Each mouse should be observed and recorded |
| | while recovering from the anesthesia. |
| | All the samples are formulated with 10 |
| | units of BoNT/A complex. |
| DAS and | DAS score analysis: Each mouse is removed |
| Rotarod | from its cage and placed on a flat surface. The |
| Analysis | mouse is then raised slightly from behind and |
| | by holding the tail, while the front legs remain |
| | on the flat surface. The technician then notes |
| | the spread of the hind limbs and toes and rates |
| | them based on the DAS scoring system. This |
| | scoring is performed at baseline before the dose |
| | administration, and at every timepoints during |
| | the study, and is recorded. Three operators |
| | will independently score each animal. |
| | DAS score study time-points: Day 1 to every |
| | other day till 45 days. |
| | Rotarod analysis: Each mouse is removed from |
| | its cage and placed on rotarod. Each mice |
| | will be recorded separately for time they spend |
| | on the rotarod and number of rotation they |
| | make before falling down. |
| | Rotarod study time-points: Time 0 before |
| | dosing, 24 hours and every other day till 45 days. |
| | Experiment repetition: twice, |
| Reporting | Raw data should be shared after each time |
| | point including image and all three independent |
| | observer data in the suggested format. |
| | Final report should be compiled and submitted |
| | in the suggested format. |
| Study | The study will be terminated upon request by |
| Termination | the sponsor, and mice will be euthanized via |
| | CO₂ inhalation. |

### Experiment:

Dose: 50 μl.
Injection volume: 100 μl.
Injection site: Both left and right foot were injected with the sample. All the dilutions were prepared in 1×PBS.
Negative control: 1×PBS.
Positive control I: In-house BoNT/A complex API (10 units): No added Hn-33.
Positive control II (FI): Evolus sample (10 units): No added Hn-33.
Sample 1 (FII): Evolus sample (10 units) mixed with (1:1; molar ratio) native Hn-33.
Sample 2 (FIII): Evolus sample (10 units) mixed with (1:3; molar ratio) native Hn-33.
Sample 3 (FIV): Evolus sample (10 units) mixed with (1:5; molar ratio) native Hn-33.
Sample 4 (FV): Evolus sample (10 units) mixed with (1:5; molar ratio) recombinant Hn-33.
Sample 5 (FVI): Evolus sample (10 units) mixed with (1:10; molar ratio) native Hn-33.
Sample 6 (FVII): In-house BoNT/A complex API sample (10 units) mixed with (1:5; molar ratio) recombinant Hn-33.

### Das Scoring Procedure:

All the observers recorded the score independently and compiled at the end of last reading. Neither the person who injected mice nor the DAS scorer knows about the sample (all the formulations were coded). Results: A) Control: 1×PBS was injected intramuscularly to both hind limb of each mouse while under anesthesia.

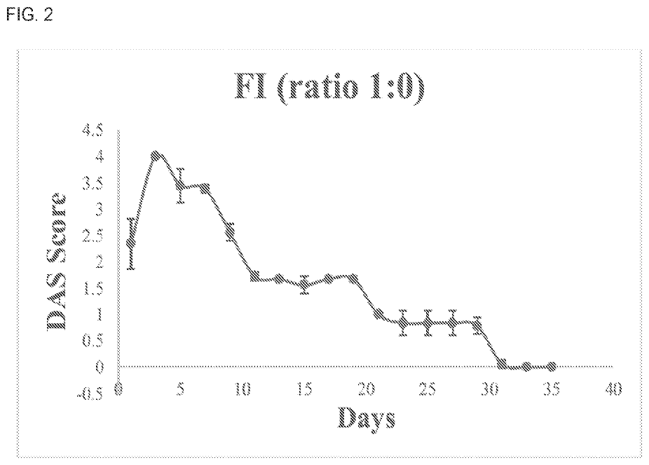

As seen in FIG. 2, Positive control II—FI (Evolus formulated BoNT/A complex: No Hn-33 added): 100 μl of 10 units of formulated BoNT/A complex (FI) was injected intramuscularly to both hind limb of each mouse while under anesthesia.

Average DAS score of both legs of all the animals in this cohort was plotted against their respective day. Error bars indicated the standard deviation of all the measurements

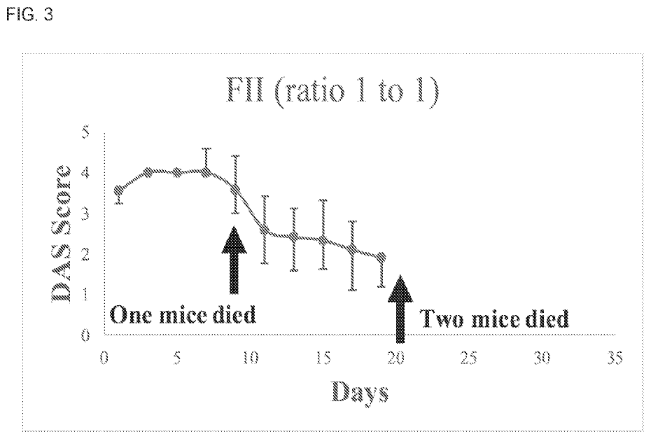

As seen in FIG. 3, Formulated complex I—FII (Evolus formulated BoNT/A complex: Hn-33 added; 1:1): Evolus sample was mixed with native Hn-33 in 1×PBS in the molar ratio of 1:1. 100 μl of 10 units of formulated BoNT/A complex (FII) was injected intramuscularly to both hind limbs of each mouse while under anesthesia.

Average DAS score of both legs of all the animals in this cohort was plotted against their respective day. Error bars indicated the standard deviation of all the measurements. All the mice died on day 20.

Thus, FIG. 3 shows the effect of augmenting a BoNT type A composition with 1M Hn-33—as compared to control (FIG. 2), the DAS score of ˜4 was maintained almost to day 10, while the DAS score of the control group had decreased below 4 by day 5.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present disclosure, which is defined solely by the claims. Accordingly, embodiments of the present disclosure are not limited to those precisely as shown and described.

Certain embodiments are described herein, comprising the best mode known to the inventor for carrying out the methods and devices described herein. Of course, variations on these described embodiments will become apparent to those of ordinary skill in the art upon reading the foregoing description. Accordingly, this disclosure comprises all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be comprised in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope otherwise claimed. No language in the present specification should be construed as indicating any non-claimed element essential to the practice of embodiments disclosed herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present disclosure so claimed are inherently or expressly described and enabled herein.

1) A composition comprising a Clostridial neurotoxin and a Clostridial NAP, wherein said NAP is present in an amount at least 10% greater than that normally associated with the toxin.

2) The composition of claim 1, wherein said NAP is present in an amount 20% greater than that normally associated with the toxin.

3) The composition of claim 2, wherein said NAP is present in an amount 40% greater than that normally associated with the toxin.

4) The composition of claim 3, wherein said NAP is present in an amount 60% greater than that normally associated with the toxin.

5) The composition of claim 1, wherein said NAP comprises a Clostridial accessory protein.

6) The composition of claim 5 wherein said accessory protein comprises Hn-33.

7) The composition of claim 1, further comprising a vasoconstrictor.

8) The composition of claim 7, wherein said vasoconstrictor comprises epinephrine.

9) The composition of claim 8, wherein said epinephrine is present in an amount of at least 0.5%.

10) A method of treating a cosmetic defect, comprising administering the composition of claim 1 to an individual.

11) The method of claim 10, wherein the cosmetic defect is a wrinkle.

12) The method of claim 11, wherein the cosmetic defect is a condition selected from the group consisting of marionette lines, glabellar lines, crows feet, brow furrows, and combinations thereof.

13) The method of claim 10, wherein said composition further comprises a vasoconstrictor.

14) The method of claim 13, wherein said vasoconstrictor comprises epinephrine.

15) A method of treating a muscle, comprising administering the composition of claim 1 to an individual.

16) The method of claim 15, wherein the total dose of the Clostridial neurotoxin is 50-200 Units.

17) The method of claim 16, wherein composition is administered by injection or topically.

18) The method of claim 17, wherein said composition is administered by injection.

19) The method of claim 17, wherein said Clostridial neurotoxin comprises BoNT/A.

20) The method of claim 17, wherein said Clostridial neurotoxin comprises BoNT/B, BoNT/C, BoNT/D, BoNT/E, BoNT/F, or BoNT/G.

21)-25) (canceled)
